(19) United States
(12) Patent Application Publication
(10) Pub. No.: US 2024/0279270 A1
(43) Pub. Date: Aug. 22, 2024

(54) METHOD FOR CARBOXYLATION OF SILK PROTEIN AND CARBOXYLATED SILK PROTEIN PREPARED BY METHOD AND APPLICATION OF CARBOXYLATED SILK PROTEIN

(71) Applicant: Westlake University, Hangzhou (CN)

(72) Inventors: Chengchen GUO, Hangzhou (CN); Ziyang SUN, Hangzhou (CN)

(21) Appl. No.: 18/566,728

(22) PCT Filed: May 17, 2022
(86) PCT No.: PCT/CN2022/093191

§ 371 Date: Dec. 4, 2023

(30) Foreign Application Priority Data
Jun. 4, 2021 (CN) 202110625401.6

(51) Int. Cl. C07K 1/107; A61L 27/22

(52) U.S. Cl. CPC C07K 1/1072; A61L 27/227

(57) ABSTRACT

The invention related to a method for carboxylating a silk fibroin, a carboxylated silk fibroin prepared therefrom and use thereof. The present invention provides a method for carboxylating silk fibroin, comprising a step of reacting a silk fibroin with a dicarboxylic anhydride in the presence of a lithium salt. Compared with the carboxylation method of silk fibroin in the prior art, the method of the present invention has the advantages of mild preparation conditions and simple preparation steps. Moreover, compared with the uncarboxylated silk fibroin, the carboxylated silk fibroin prepared in the present invention has unreduced molecular weight with increased hydrophilicity.

### CROSS-REFERENCE TO RELATED APPLICATION

This application claims benefits of the priority of Chinese invention patent application No. 202110625401.6 filed with the China National Intellectual Property Administration on Jun. 4, 2021, the entire contents of which are incorporated herein by reference to the extent that they are recorded herein.

### TECHNICAL FIELD

The present invention relates to the technical field of modification of fibroin-based natural polymer materials, and in particular to a method for carboxylating silk fibroin and the carboxylated silk fibroin prepared therefrom and use thereof.

### BACKGROUND

Protein-based natural polymer materials, such as collagen, elastin, silk fibroin, etc., are widely used in implanted interventional materials, tissue repair, tissue engineering, and drug sustained release and other biomedicine and bioengineering fields due to their good biocompatibility and biodegradability. However, compared with synthetic polymer materials, the chemical modification methods that have been developed for protein-based natural polymer materials are very limited, and it is difficult to prepare protein-based materials with high modification and controllable modification rate. The lack of an efficient chemical modification method greatly hinders the practical application and future development of protein-based natural polymer materials.

Silk fibroin, mainly derived from silk, is a structural protein that makes up the silk. Due to its excellent mechanical properties, biocompatibility and biodegradability, it has attracted widespread attention in recent years, especially in the biomedical field. For example, silk fibroin films can be used as substrates for preparing biosensors; silk fibroin sponges can be used as tissue engineering scaffolds; silk fibroin nanospheres can be used as carriers for drug delivery and release; silk fibroin blocks can be processed into implantable bone nails, etc. Silk fibroin can be extracted from natural silkworm cocoons through dissolution and regeneration, which method is green and environmentally friendly and has good practical application value.

Silk fibroin in natural silkworm silk is mainly composed of heavy chains and light chains, with molecular weights of 390 kDa and 26 kDa, respectively. The heavy chain and light chain are connected by disulfide bonds. The main amino acids and their proportions in silk fibroin are glycine (42.9%), alanine (30%), serine (12.2%), tyrosine (4.8%), valine (2.5%), aspartic acid and asparagine (2%), etc. The heavy chain is mainly composed of 12 domains, which contain highly repeated amino acid sequences, forming a semi-crystalline structure. At the molecular level, it consists of nanocrystals formed by β-sheet structures (hereinafter referred to as β-sheet nanocrystals) embedded in an amorphous continuous phase with lower crystallinity. Natural silk fibroin will form a highly disordered structure in the solution after degumming, dissolving, and purifying. By freeze-drying, silk fibroin with highly disordered structures in an aqueous solution can be prepared into silk fibroin powders with amorphous morphology. The silk fibroin powder can be used as an additive in some skin care products or cosmetics.

As a structural protein, natural silk fibroin is mainly characterized by excellent mechanical properties, good biocompatibility, and good biodegradability. However, its biological functions are limited, especially it is lack of bio-respondence. Therefore, there is an urgent need to construct silk fibroin with specific biological functions and bio-responsiveness, and it is also a key step in promoting the application of silk fibroin in more fields, especially in the biomedical field.

Chemical modification of silk fibroin mainly involves chemical reactions of the side chain functional groups (such as hydroxyl, amino, etc.) of the silk fibroin molecular chain, among which the carboxylation of silk fibroin is a research focus.

Carboxylated silk fibroin has good application prospects since functionalization of silk fibroin can be achieved by converting the hydroxyl groups of the side chains of the silk fibroin molecular chains into carboxyl groups through specific chemical reactions and then incorporating active molecule fragments or drugs at the carboxyl sites by click chemistry methods.

Attempts had been made to convert the hydroxyl group of the side chain into a carboxyl group through the substitution reaction of chloroacetic acid or the oxidation reaction of sodium hypochlorite (Kaplan D. L. et al. Biomacromolecules 2016, 17, 237; Kaplan D. L. et al. Biomacromolecules 2020, 21, 2829; Fan Y. et al. ACS Appl. Mater. Interfaces 2016, 8, 14406.), but the reaction conditions were relatively harsh and would affect other chemical structures of the silk fibroin, resulting in a decrease in molecular weight of the silk fibroin. In addition, an ionic liquid system was used to carboxylate silk fibroin (Burke K. A. et al. Bioconjugate Chem. 2020, 31, 1307-1312.), but the cost of ionic liquid is high such that the method is not economical. Therefore, there remains a need to develop an economical, low-cost, and efficient method for carboxylation modification of silk fibroin.

### SUMMARY OF THE INVENTION

One object of the present invention is to provide a method for carboxylating a silk fibroin.

Another object of the present invention is to provide a carboxylated silk fibroin prepared by the method.

Another object of the present invention is to provide the use of carboxylated silk fibroin in medical engineering materials.

In one aspect, the present invention provides a method for carboxylating a silk fibroin, which comprises reacting a silk fibroin with a dicarboxylic anhydride in the presence of a lithium salt.

More specifically, the method comprises the following steps:
(1) reacting a silk fibroin with a dicarboxylic anhydride in the presence of a lithium salt;
(2) dialyzing the reaction product in step (1) to obtain an aqueous solution of a carboxylated silk fibroin and drying the solution (for example, freeze-drying) to obtain the carboxylated silk fibroin.

In the present invention, the silk fibroin refers to a material based on silk fibroin, such as a natural silk fibroin, a recombinant silk fibroin, a regenerated silk fibroin with different molecular weights, etc.

In some embodiments, the silk fibroin is prepared by steps of:
1′: adding silkworm cocoons to an aqueous solution of sodium carbonate, heating the solution to boiling, and maintaining it for 30 to 120 minutes to obtain a degummed silk, rinsing the degummed silk in water several times and then drying the same at room temperature;
2′: adding the degummed silk to an aqueous solution of lithium bromide, heating the solution to dissolve the silk to obtain a solution, and dialyzing the solution to obtain a silk fibroin solution.

The silk fibroin solution prepared in the above step 2′ can be directly used in the carboxylation process of the present invention or can be dried (such as freeze-dried) to obtain a silk fibroin solid, which is then used in the carboxylation process.

In some embodiments, the silk fibroin is reacted with the dicarboxylic anhydride in a solvent, and the solvent may be an aprotic polar solvent such as dimethyl sulfoxide, dimethylformamide, dimethylacetamide, methylpyrrolidone, etc. In the reaction solution, the concentration of lithium ions may be 0.5 to 1.5 mol/L, such as 0.5, 0.6, 0.7, 0.8, 0.9, 1.0, 1.1, 1.2, 1.3, 1.4, 1.5 mol/L, etc., and the concentration of the silk fibroin may be 1 to 20 g/L, such as 2, 3, 5, 7.5, 10, 15, 20 g/L, etc.

In some embodiments, the dicarboxylic anhydride may be selected from succinic anhydride, glutaric anhydride, phthalic anhydride, and the like.

In some embodiments, the lithium salt is lithium chloride or lithium bromide, particularly lithium chloride. In the case of using lithium chloride, silk fibroin powder can be better dissolved in the solution.

In some embodiments, the mass ratio of the silk fibroin to the dicarboxylic anhydride may be 1:0.1 to 1:10, preferably 1:1 to 1:10, more preferably 1:2 to 1:10, especially 1:5 to 1:10, such as 1:5, 1:6, 1:7, 1:7.5, 1:8, 1:9, 1:10, etc. The carboxylation modification rate of serine/tyrosine of the carboxylated silk fibroin can be controlled by adjusting the mass ratio of the silk fibroin powder to the dicarboxylic anhydride.

In some embodiments, the silk fibroin may be reacted with the dicarboxylic anhydride at 20 to 60° C., preferably at 40 to 50° C. For example, the reaction may be carried out at 25° C., 35° C., 40° C., 45° C., or 50° C.

In some embodiments, the silk fibroin may be reacted with the dicarboxylic anhydride for 5 minutes to 72 hours, preferably for 0.5 to 6 hours, for example, for 0.5 hours, 1 hour, 2 hours, 4 hours, or 6 hours.

In yet another aspect, the present invention provides a carboxylated silk fibroin prepared by the above method.

In particular, in the carboxylated silk fibroin, a hydroxyl group of serine and/or tyrosine of the silk fibroin reacts with a dicarboxylic anhydride to form a structure of formula I:

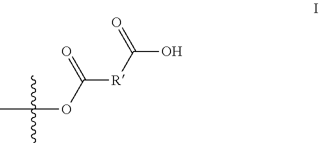

wherein in Formula I, R′ is a C2-C6 hydrocarbonylene group, which is determined by the structure of the dicarboxylic anhydride used in the above preparation method.

In some embodiments, in Formula I, R′ is independently ethylene, propylene, or 1,2-phenylene.

In some embodiments, in the carboxylated silk fibroin, serine is modified by 20 to 90%, and tyrosine is modified by 10 to 35%.

In yet another aspect, the present invention provides a use of the above-mentioned carboxylated silk fibroin in preparing a medical bioengineering material.

In another aspect, the present invention provides a biological article prepared from the carboxylated silk fibroin. Specifically, the article is in the form of a porous scaffold, film or hydrogel.

In some embodiments, the porous scaffold is prepared by steps of:
1′: dissolving the above carboxylated silk fibroin in water to obtain a carboxylated silk fibroin solution;
2′: adding the carboxylated silk fibroin solution into a mold and freeze-drying the same to obtain the carboxylated silk fibroin porous scaffold.

In some embodiments, the film is prepared by steps of:
1′: dissolving the above carboxylated silk fibroin in water to obtain a carboxylated silk fibroin solution;
2′: coating the carboxylated silk fibroin solution in the mold and air drying the same to obtain a carboxylated silk fibroin film.

In some embodiments, the hydrogel is prepared by steps of:
1′: dissolving the above carboxylated silk fibroin in water to obtain a carboxylated silk fibroin solution;
2′: adding the carboxylated silk fibroin solution into a mold, and adding peroxidase and hydrogen peroxide thereto such that the tyrosine residues in the carboxylated silk fibroin undergo oxidative cross-linking reaction in the presence of the peroxidase and hydrogen peroxide to obtain the carboxylated silk fibroin hydrogel.

In some embodiments, in the above step 1′ for preparing each of the aforementioned biological articles, the concentration of the carboxylated silk fibroin solution may be 5 to 100 g/L, such as 5, 10, 20, 30, 40, 60, 80, 100 g/L.

Unless otherwise indicated, numerical values in this disclosure represent approximate measures or limitations of the ranges of embodiments that include minor deviations from the given values and have about the values recited as well as having the precise value recited. Except the detailed description of the last embodiment, all numerical values for parameters (eg, quantities or conditions) in this application document (including the appended claims) are to be understood in all cases as modified by the term “about”, regardless of whether “approximately” actually appears before the value. “Approximately” means that the stated value is allowed to be slightly less precise (somewhere near the exact value; about or reasonably close to the value; approximation). If the imprecision resulted from “approximately” causes that there is no common meaning in the art, “approximately” as used herein at least means the variation that can be produced by ordinary methods for measuring and using these parameters. For example, “about” may include variations of less than or equal to 15%, less than or equal to 10%, less than or equal to 5%, less than or equal to 4%, less than or equal to 3%, less than or equal to 2%, less than or equal to 1%, less than or equal to 0.5%, less than or equal to 0.1%, and in some aspect, a variation of less than or equal to 0.01%.

### Beneficial Effects

(1) The silk fibroin required for the method of the present invention is directly extracted from silkworm cocoons, which is widely available, cheap, and easy to obtain; dicarboxylic anhydrides, such as succinic anhydride, glutaric anhydride, phthalic anhydride, etc., are common Industrial raw materials at low prices. The preparation steps of the method involve mild conditions and simple operations and are very suitable for mass production.
(2) The degree of carboxylation of the carboxylated silk fibroin obtained by the method of the present invention can be accurately adjusted through reaction conditions such as reactant concentration, ratio of the reactants, reaction temperature, and reaction time.
(3) The carboxylated silk fibroin obtained by the method of the present invention has better hydrophilicity than the unmodified silk fibroin. The carboxyl functional groups in the molecule can be modified into other functional groups through chemical reactions.
(4) The method of the present invention can be applied to a variety of silk fibroin, such as natural silk fibroin, recombinant silk fibroin, and regenerated silk fibroin with different molecular weights, etc.
(5) The carboxylated silk fibroin-based material of the present invention has low cytotoxicity and good biocompatibility and can be widely used in medical and bioengineering materials.

### DESCRIPTION OF DRAWINGS

FIG. 1 shows a photograph of the carboxylated silk fibroin powder prepared in Example 1 of the present invention.

FIG. 2 shows the hydrogen NMR spectrum in deuterated dimethyl sulfoxide of the carboxylated silk fibroin prepared in Example 1 of the present invention.

FIG. 3 shows the hydrogen NMR spectrum in deuterated dimethyl sulfoxide of the silk fibroin raw material used in the example of the present invention.

FIG. 4 shows the infrared absorption spectrum of the carboxylated silk fibroin prepared in Example 1 of the present invention.

FIG. 5 shows the hydrogen NMR spectrum in deuterated dimethyl sulfoxide of the carboxylated silk fibroin prepared in Example 2 of the present invention.

FIG. 6 shows the infrared absorption spectrum of the carboxylated silk fibroin prepared in Example 2 of the present invention.

FIG. 7 shows the polyacrylamide gel electrophoresis results of the silk fibroin raw material and the carboxylated silk fibroin prepared in Example 1 of the present invention.

FIG. 8 shows the contact angle test results of the silk fibroin raw material and the carboxylated silk fibroin prepared in Example 1 of the present invention.

FIG. 9 shows a photograph of the carboxylated silk fibroin film prepared in Example 12 of the present invention.

FIG. 10 shows the Masson trichrome staining picture of tissue sections obtained from the back of mice which has been implanted with the carboxylated silk fibroin film into the back for 30 days in Example 13 of the present invention.

FIG. 11 shows the cell morphology of NIH-3T3 cells after being incubated on the film for 24 hours in Example 14 of the present invention.

FIG. 12 shows a photograph of the carboxylated silk fibroin porous scaffold prepared in Example 15 of the present invention.

FIG. 13 shows a photograph of the carboxylated silk fibroin hydrogel prepared in Example 16 of the present invention.

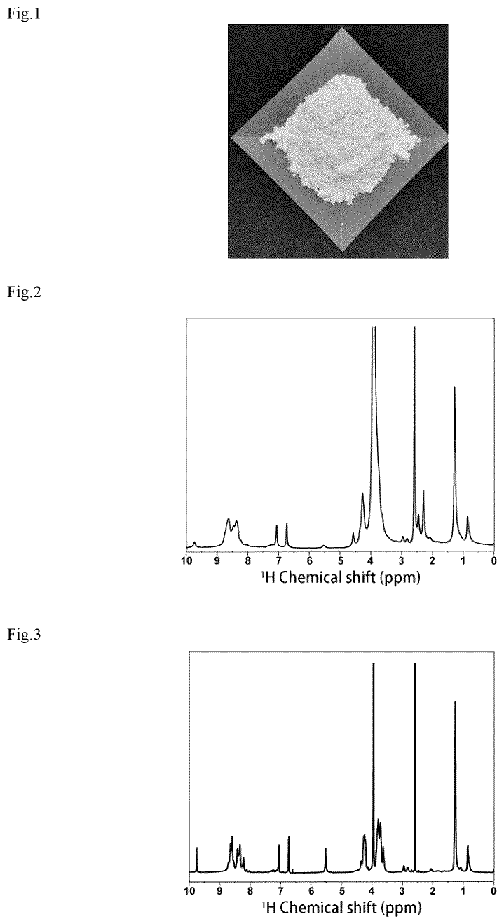

### DETAILED EMBODIMENTS

The preparation method of the present invention is further described in details with reference to the following examples. The examples are only for illustration and do not limit the scope of the invention in any way.

Materials: The silkworm cocoons used in the preparation examples were purchased from local manufacturers in Hangzhou. Lithium chloride, dimethyl sulfoxide, and succinic anhydride used in the examples were purchased from J&K Scientific, Ltd. and used as it is. BALB/c mice were provided by GemPharmatech Co., Ltd. Jiangsu, and NIH-3T3 cells were provided by BeNa Biotechnology Co., Ltd.

Hydrogen nuclear magnetic spectrum test was performed on a Bruker AVANCE NEO 600 MHz nuclear magnetic resonance spectrometer with a solvent of deuterated dimethyl sulfoxide containing lithium chloride in a concentration of 1 mol/L.

Infrared spectroscopy test was performed on a Thermo Nicolet iS50 infrared spectrometer with a scan number of 64 and a resolution of 4 cm⁻.

The contact angle test was performed on an optical contact angle measuring device of dataphysics OCA25, with a droplet volume of 5 μL.

### PREPARATION EXAMPLE

Silk fibroin was prepared as follows.
1′: Silkworm cocoons were added to an aqueous solution with a sodium carbonate concentration of 20 mmol/L, heated to boiling and kept for 30 to 120 minutes. The degummed silk was rinsed several times in water and then dried at room temperature.
2′: The degummed silk was added to an aqueous solution with a lithium bromide concentration of 9.3 mol/L, heated to 60° C. and kept for 4 hours. After the silk was dissolved, the obtained solution was dialyzed to obtain a silk fibroin solution.
3′: The silk fibroin solution was freeze-dried and then ground to obtain a silk fibroin, which was used in the subsequent carboxylation process.

### Example 1

(1) 1 g of the silk fibroin prepared in the above preparation example was dissolved in a dimethyl sulfoxide solution (50 mL) with a lithium chloride concentration of 1 mol/L, 10 g of succinic anhydride was added thereto, and the resultant was heated to 50° C. to react for 6 hours.
(2) The solution after the reaction was dialyzed to obtain an aqueous solution of carboxylated silk fibroin, which was freeze-dried to obtain a carboxylated silk fibroin dry powder.

The yield of the prepared carboxylated silk fibroin was 86% by weighing, and its hydrogen NMR spectrum in deuterated dimethyl sulfoxide is shown in FIG. 2.

The peaks at chemical shifts of 5.5 and 9.7 ppm in the hydrogen NMR spectrum correspond to the NMR signals of the hydrogen atoms in the hydroxyl groups of serine and tyrosine, respectively. Compared with the hydrogen NMR spectrum of the silk fibroin raw material (FIG. 3), it can be seen that the intensities of these two peaks are significantly reduced, indicating that the hydroxyl group has undergone an esterification reaction. By comparing the peak areas, it can be calculated that the carboxylated serine in the prepared silk fibroin molecule accounts for 83.7% of the serine in the silk fibroin molecule before reaction, and the carboxylated tyrosine accounts for 28.5% of the tyrosine in the silk fibroin molecule before reaction.

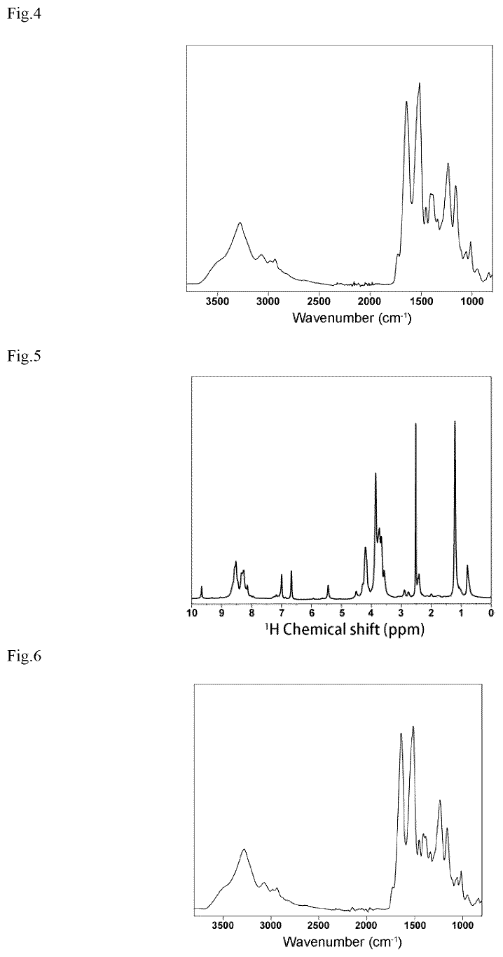

The obtained infrared absorption spectrum of the carboxylated silk fibroin is shown in FIG. 4.

### Example 2

A carboxylated silk fibroin was prepared in the same manner as that in Example 1, except that 1 g of succinic anhydride was used.

The hydrogen NMR spectrum of the prepared carboxylated silk fibroin in deuterated dimethyl sulfoxide is shown in FIG. 5, and the infrared absorption spectrum is shown in FIG. 6.

### Example 3

A carboxylated silk fibroin was prepared in the same manner as that in Example 1, except that the reaction temperature was set at 25° C.

### Example 4

A carboxylated silk fibroin was prepared in the same manner as that in Example 1, except that the reaction temperature was set at 40° C.

### Example 5

A carboxylated silk fibroin was prepared in the same method as that in Example 1, except that 1 g of the silk fibroin prepared in the preparation example was dissolved in a dimethyl sulfoxide solution (100 mL) with a lithium chloride concentration of 1 mol/L.

### Example 6

A carboxylated silk fibroin was prepared in the same manner as that in Example 1, except that the reaction time was 0.5 h.

### Example 7

A carboxylated silk fibroin was prepared in the same manner as that in Example 1, except that the reaction time was 2 h.

### Example 8

A carboxylated silk fibroin was prepared in the same manner as that in Example 1, except that the reaction time was 24 h.

### Example 9

A carboxylated silk fibroin was prepared in the same manner as that in Example 1, except that 2.5 g of succinic anhydride was used.

### Example 10

A carboxylated silk fibroin was prepared in the same manner as that in Example 1, except that 5 g of succinic anhydride was used.

### Example 11

A carboxylated silk fibroin was prepared in the same method as that in Example 1, except that a dimethylformamide solution (50 mL) with a lithium chloride concentration of 1 mol/L was used.

### Example 12

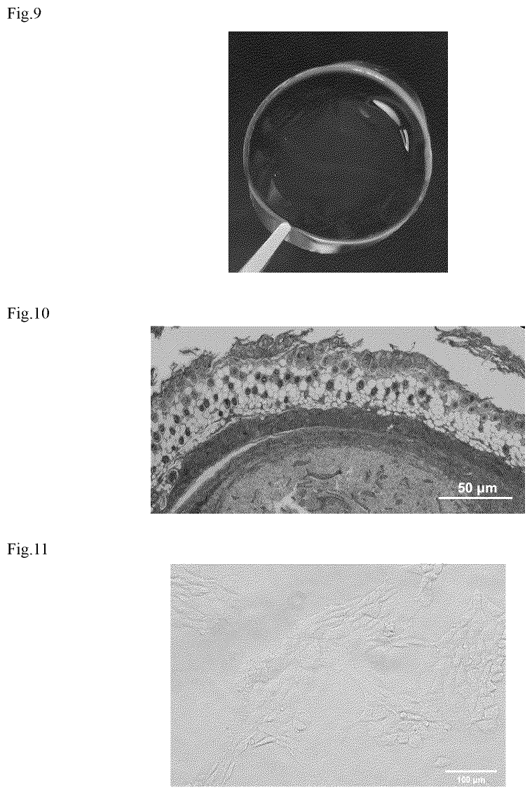

40 mg of the carboxylated silk fibroin powder prepared in Example 1 was dissolved in 1 mL of pure water to obtain a carboxylated silk fibroin solution with a concentration of 40 mg/mL. The solution was applied to a mold and dried naturally at room temperature to obtain a carboxylated silk fibroin film with a thickness of about 80 μm, whose photo is shown in FIG. 9. The film can be used in biomedical applications such as tissue repair and two-dimensional cell culture.

### Example 13

5 mg of the carboxylated silk fibroin film in Example 12 was immersed in methanol for 3 hours, and then rinsed several times with PBS buffer to remove excess methanol, sterilized under ultraviolet irradiation, and implanted subcutaneously in the back of BALB/c mice. After 30 days of implantation, it was taken out and the biocompatibility of the carboxylated silk fibroin material was analyzed with tissue sections, whose photo is shown in FIG. 10. The thickness of the fibrosis tissue layer was measured to be about 50 μm, showing that the carboxylated silk fibroin film did not promote the generation of obvious fibrosis tissue in mice, indicating that it has good biocompatibility.

### Example 14

2 mg of the carboxylated silk fibroin powder in Example 1 was dissolved in 0.1 mL of pure water to obtain a carboxylated silk fibroin solution with a concentration of 20 mg/mL. The solution was applied to the wells of a 48-well cell culture plate and dried naturally at room temperature to obtain a carboxylated silk fibroin film with a thickness of approximately 10 μm. After being immersed in methanol for 3 hours, the carboxylated silk fibroin film was rinsed several times with PBS buffer to remove excess methanol. After sterilization under ultraviolet irradiation, the film was added with 20 μL of a NIH-3T3 cell suspension (1*10⁶ cells/mL) and 200 μL of a DMEM culture medium and incubated at 37° C. for 24 hours. The cytocompatibility of the carboxylated silk fibroin material was analyzed by observing the cell growth under a microscope, and the photo is shown in FIG. 11. As shown in FIG. 11, NIH-3T3 cells can adhere to the film and grow normally, indicating that the film has good cytocompatibility.

### Example 15

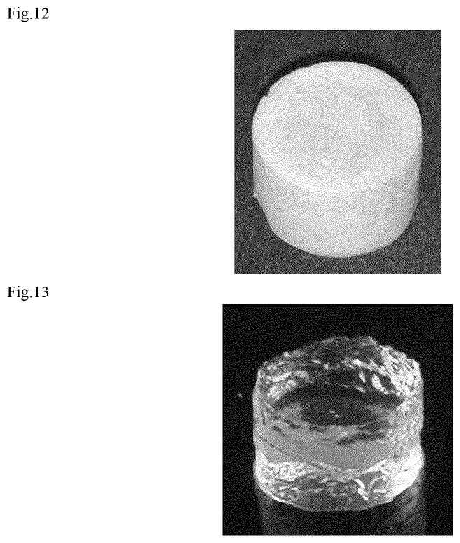

40 mg of the carboxylated silk fibroin powder in Example 1 was dissolved in 1 mL of pure water to obtain a carboxylated silk fibroin solution with a concentration of 40 mg/mL. The solution was added to a mold and then freeze-dried to obtain a carboxylated silk fibroin porous scaffold, whose photo is shown in FIG. 12. The porous scaffold can be used for biomedical applications such as tissue repair and three-dimensional cell culture.

### Example 16

20 mg of the carboxylated silk fibroin powder in Example 1 was dissolved in 1 mL of pure water to obtain a carboxylated silk fibroin solution with a concentration of 20 mg/mL. 10 μL of a 1% hydrogen peroxide solution and 10 μL of a peroxidase solution (1000 U/mL) were added to the solution, and the resultant was incubated at 37° C. to obtain a carboxylated silk fibroin hydrogel, whose photo is shown in FIG. 13. The hydrogel can be used in biomedical applications such as tissue repair and drug delivery.

Table 1 below shows the preparation conditions of Examples 1-11 and the serine/tyrosine modification rates of the prepared products.

TABLE 1

| | Reac- | Concen- | | Mass ratio | | |
|---|---|---|---|---|---|---|
| | tion | tration | Reac- | of silk | Serine | Tyrosine |
| | temper- | of Silk | tion | fibroin to | modifica- | modifica- |
| Exam- | ature | fibroin | time | dicarboxylic | tion rate | tion rate |
| ple | (° C.) | (g/L) | (h) | anhydride | (%) | (%) |
| 1 | 50 | 20 | 6 | 1:10 | 83.7 | 28.5 |
| 2 | 50 | 20 | 6 | 1:1 | 43.5 | 22.9 |
| 3 | 25 | 20 | 6 | 1:10 | 52.9 | 18.7 |
| 4 | 40 | 20 | 6 | 1:10 | 67.3 | 27.8 |
| 5 | 50 | 10 | 6 | 1:10 | 63.6 | 22.1 |
| 6 | 50 | 20 | 0.5 | 1:10 | 26.3 | 15.3 |
| 7 | 50 | 20 | 2 | 1:10 | 62.7 | 19.5 |
| 8 | 50 | 20 | 24 | 1:10 | 81.4 | 33.2 |
| 9 | 50 | 20 | 6 | 1:2.5 | 53.2 | 23.4 |
| 10 | 50 | 20 | 6 | 1:5 | 68.4 | 25.5 |
| 11 | 50 | 20 | 6 | 1:10 | 67.6 | 28.1 |

As shown in Table 1 above, the modification rate of serine and tyrosine can be increased by extending the reaction time, increasing the reaction temperature, or increasing the mass ratio of dicarboxylic anhydride to silk fibroin powder.

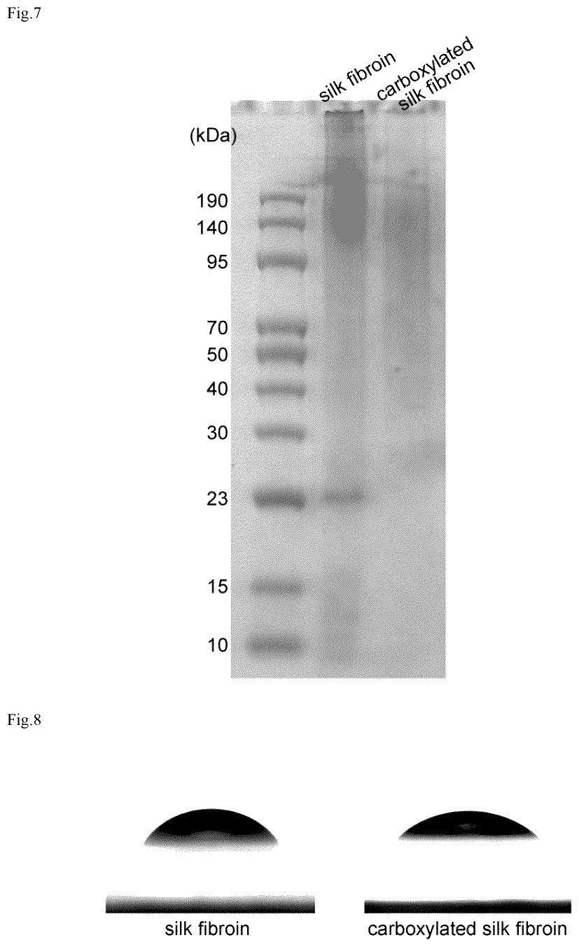

As shown in FIG. 7, the molecular weight of the prepared carboxylated silk fibroin is basically consistent with the molecular weight of the silk fibroin raw material, confirming that this carboxylation reaction will not cause a decrease in fibroin molecular weight.

As shown in FIG. 8, the prepared carboxylated silk fibroin film has a smaller contact angle than the unmodified silk fibroin film (45° for carboxylated silk fibroin film, 590 for unmodified silk fibroin film), indicating that the carboxylated silk fibroin has better hydrophilicity. Such improved hydrophilicity is beneficial for developing the carboxylated silk fibroin of the present application into a biomaterial with specific fibroin adsorption and cell adsorption properties, and thus the material has application values in fields of tissue repair and cell culture.

### Comparative Example 1

In Biomacromolecules 2016, 17, 237 and Biomacromolecules 2020, 21, 2829, a 10 mol/L sodium hydroxide solution was added to a silk fibroin solution with a concentration of 45 mg/mL, and diluted to a final concentration of sodium hydroxide of 3 mol/L. Then, chloroacetic acid (1 mol/L) was added and reacted at room temperature for 1 hour. After the reaction was terminated by adding a solution of monosodium phosphate, the reaction solution was neutralized to pH 7.4 with 10 mol/L hydrochloric acid. An ice bath was used for cooling during the neutralization process. Finally, the reacted solution was dialyzed to obtain an aqueous solution of a carboxylated silk fibroin.

According to the experimental results in the literature, the peak molecular weight of the silk fibroin molecules was reduced from the original 131.3 kDa to 36.4 kDa after the carboxylation reaction. Moreover, the serine modification rate in this carboxylation reaction was only 19.9%.

### Comparative Example 2

In ACS Appl. Mater. Interfaces 2016, 8, 14406, a certain amount of a sodium hypochlorite solution (0 to 5 mmol of sodium hypochlorite per gram of silk fibroin) was added to a silk fibroin solution, and a sodium hydroxide solution was added to adjust the pH of the solution to 10, and the pH value was maintained by adding the sodium hydroxide solution from time to time. The reaction was carried out at room temperature. After the reaction was completed, hydrochloric acid was added to adjust the pH to 7, and finally an aqueous solution of carboxylated silk fibroin was obtained after dialysis.

According to the experimental results in the literature, the serine modification rate was approximately 47% under optimal conditions. The change in the molecular weight of the product was not characterized in the literature, but the product yield was significantly reduced at a high sodium hypochlorite ratio, and it is inferred that the oxidation reaction of sodium hypochlorite would cause a decrease in the molecular weight of the silk fibroin.

1. A method for carboxylating a silk fibroin, comprising a step of reacting the silk fibroin with a dicarboxylic anhydride in the presence of a lithium salt.

2. The method of claim 1, wherein the method comprises the steps of:
(1) Reacting a silk fibroin with a dicarboxylic anhydride in the presence of a lithium salt;
(2) Dialyzing the reaction product obtained in step (1) to obtain an aqueous solution of a carboxylated silk fibroin and drying the solution to obtain the carboxylated silk fibroin.

3. The method of claim 1, wherein the dicarboxylic anhydride is selected from succinic anhydride, glutaric anhydride, and phthalic anhydride.

4. The method of claim 1, wherein the silk fibroin is prepared by the steps of:
1′: Adding silkworm cocoons to an aqueous solution of sodium carbonate, heating the solution to boiling, and maintaining it for 30 to 120 minutes to obtain a degummed silk, rinsing the degummed silk in water several times and then drying the same at room temperature;
2′: Adding the degummed silk to an aqueous solution of lithium bromide, heating the solution to dissolve the silk to obtain a solution, and dialyzing the solution to obtain a silk fibroin solution.

5. The method of claim 1, wherein,
the silk fibroin is reacted with the dicarboxylic anhydride in a solvent and/or the concentration of lithium ion is 0.5 to 1.5 mol/L, and/or
the concentration of the silk fibroin is 1 to 20 g/L, and/or
the mass ratio of the silk fibroin to the dicarboxylic anhydride is from 1:0.1 to 1:10.

6. The method of claim 5, wherein, the solvent is dimethyl sulfoxide,
dimethylformamide, dimethylacetamide, or methylpyrrolidone, and/or
the mass ratio of the silk fibroin to the dicarboxylic anhydride is from 1:1 to 1:10.

7. The method of claim 1, wherein, the lithium salt is lithium chloride or lithium bromide, preferably lithium chloride.

8. The method of claim 1, wherein, the silk fibroin is reacted with dicarboxylic anhydride at 20 to 60° C., and/or the silk fibroin is reacted with the dicarboxylic anhydride for 0.5 to 72 hours.

9. The method of claim 1, wherein, the silk fibroin is reacted with dicarboxylic anhydride at 40 to 50° C., and/or the silk fibroin is reacted with the dicarboxylic anhydride for 0.5 to 6 hours.

10. A carboxylated silk fibroin, which is prepared by the method of claim 1, wherein, in the carboxylated silk fibroin, a hydroxyl group of serine and/or tyrosine of the silk fibroin react with a dicarboxylic anhydride to form a structure of formula I: 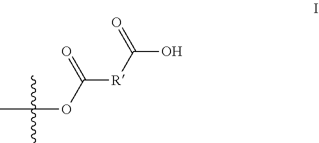
in Formula I, R′ is a C2-C6 hydrocarbonylene group, which is determined by the structure of the dicarboxylic anhydride used in the carboxylation process.

11. The carboxylated silk fibroin of claim 10, wherein,
in Formula I, R′ is independently ethylene, propylene, or 1,2-phenylene, and/or in the carboxylated silk fibroin, the serine is modified by 20 to 90%, and the tyrosine is modified by 10 to 35%.

12. A method for preparing a medical bioengineering material, comprising the step of using the carboxylated silk fibroin of claim 10 as a raw material.

13. A biological article prepared from the carboxylated silk fibroin of claim 10.

14. The biological article of claim 13, wherein, the article is a porous scaffold, a film or a hydrogel.

15. The biological article of claim 14, wherein, the porous scaffold is prepared from the carboxylated silk fibroin via a solution freeze-drying method.

16. The biological article of claim 14, wherein, the film is prepared from the carboxylated silk fibroin via a coating method.

17. The biological article of claim 14, wherein, the hydrogel is prepared from the carboxylated silk fibroin via an enzyme cross-linking method.

18. The biological article of claim 17, wherein, the hydrogel is prepared by a method comprising: adding a solution of the carboxylated silk fibroin into a mold, adding peroxidase and hydrogen peroxide thereto to perform the enzyme cross-linking method.
